SEQUENCE LISTING (1 sequence)

SEQ ID NO: 1 - DNA, 36 bp, Artificial Sequence
  Colorectal Cancer Cell Targeting Ligand
acgtggtataaaatcgcgtttcagcgcaaccgaaaa

(19) United States
(12) Patent Application Publication
(10) Pub. No.: US 2024/0218368 A1
(43) Pub. Date: Jul. 4, 2024

(54) COLON CANCER-SPECIFIC TARGET EXOSOME COMPOSITION AS DRUG DELIVERY MATERIAL, AND USE THEREOF

(71) Applicants: THE CATHOLIC UNIVERSITY OF KOREA INDUSTRY-ACADEMIC COOPERATION FOUNDATION, Seoul (KR); MYONGJI HOSPITAL, Gyeonggi-do (KR)

(72) Inventors: Say June KIM, Seoul (KR); Ok Hee KIM, Chungcheongnam-do (KR); Min Ki KIM, Gyeonggi-do (KR)

(21) Appl. No.: 18/558,075

(22) PCT Filed: Mar. 4, 2022
(86) PCT No.: PCT/KR2022/003121

§ 371 Date: Oct. 30, 2023

(30) Foreign Application Priority Data
Apr. 30, 2021 (KR) 10-2021-0056790

(51) Int. Cl. C12N 15/113; A61K 9/50; A61K 9/51; A61P 35/00; C12N 5/077; C12N 15/85

(52) U.S. Cl. CPC C12N 15/1135; A61K 9/5063; A61K 9/5192; A61P 35/00; C12N 5/0653; C12N 15/85; C12N 2310/14

(57) ABSTRACT

The present invention relates to: a pharmaceutical composition comprising extracellular vesicles transformed with a recombinant expression vector in which a gene expressing a ligand that binds to receptors of colorectal cancer cells is inserted into a gene that is expressed in extracellular vesicles of stem cells; and a method for preparing an expression vector for preventing or treating colorectal cancer. The present invention has an advantage in that exosomes, as extracellular vesicles, can be utilized as a drug delivery material and target and specifically act on colorectal cancer by selectively binding to colorectal cancer cells which are target cells.

### TECHNICAL FIELD

The present invention relates to a colorectal cancer-specific target exosome composition comprising exosomes transformed from a recombinant expression vector and a use thereof.

### BACKGROUND ART

The large intestine is roughly divided into the colon and the rectum, cancer occurring in the colon is colon cancer, and cancer occurring in the rectum is rectal cancer, which are also commonly called large intestine cancer or colorectal cancer. For colorectal cancer, almost all cancers of the large intestine and rectum (colorectal) are adenocarcinomas, which develop from the lining of the large intestine (colon) and rectum, the cancer begins by growing on the surface of the lining of the bowel or rectum and can invade nearby lymph nodes, and the incidence of colorectal cancer is characterized by a sharp increase around the age of 40 to 50.

Exosomes are only 30 to 100 nm in size, but they are materials that contain various materials such as proteins, nucleic acids, and lipids in the original cells, and are nanoparticles secreted by our adipose-derived stem cells such as immune cells and stem cells. Exosomes are attracting attention as next-generation anticancer materials as they are known to function as intercellular communication mediators.

Therefore, the present inventors have made extensive efforts to develop colorectal cancer-specific target extracellular vesicles as a drug delivery material, and as a result, completed the present invention by confirming that exosomes extracted from adipose-derived stem cells have the function of an anticancer drug targeting colorectal cancer.

### DISCLOSURE

### Technical Problem

An object of the present invention is to provide a pharmaceutical composition for preventing or treating colorectal cancer, comprising extracellular vesicles transformed with a recombinant expression vector in which a gene expressing a ligand that binds to receptors of colorectal cancer cells is inserted into a gene that is expressed in extracellular vesicles of stem cells.

Another object of the present invention is to provide a method for preparing a pharmaceutical composition for preventing or treating colorectal cancer, the method comprising: (a) introducing a gene expressing a ligand that binds to receptors of colorectal cancer cells into a gene that is expressed in extracellular vesicles of adipose-derived stem cells: (b) obtaining extracellular vesicles by culturing the gene-introduced cells in a medium; and (c) transfecting the resulting extracellular vesicles with a nucleic acid capable of suppressing the expression of a gene inducing colorectal cancer.

### Technical Solution

Each description and embodiment disclosed in the present application may also be applied to other descriptions and embodiments. That is, all combinations of various elements disclosed in this application fall within the scope of the present application. Further, the scope of the present application is not limited by the specific description described below.

An aspect of the present invention provides a pharmaceutical composition for preventing or treating colorectal cancer, comprising extracellular vesicles transformed with a recombinant expression vector in which a gene expressing a ligand that binds to receptors of colorectal cancer cells is inserted into a gene that is expressed in extracellular vesicles of stem cells.

The “expression vector” in the present invention may be selected from the group consisting of a linear DNA vector, a plasmid DNA vector and a recombinant viral vector, but is not limited thereto, and all typical vectors used for transformation in the art may be used in the method of the present invention. In an exemplary embodiment of the present invention, the expression vector may be a pDisplay vector.

The “extracellular vesicles” in the present invention refer to a small sphere surrounded by a cell-derived membrane, and the extracellular vesicles may be extracellular vesicles derived from nature such as plants, animals, microorganisms, or artificially produced extracellular vesicles. Further, the cells may be cells isolated from natural organisms.

The “extracellular vesicles” may be exosomes, ectosomes or microvesicles, but in an exemplary embodiment of the present invention, the “extracellular vesicles” may be exosomes.

The “exosomes” in the present invention are small vesicles having a membranous structure secreted from various cells, and refer to vesicles released into the extracellular environment after the fusion of a multivesicular body and a cytoplasmic membrane. In an exemplary embodiment of the present invention, the exosomes may be derived or isolated from adipose-derived stem cells.

In an exemplary embodiment of the present invention, the exosomes may have a diameter of 50 to 150 nm. In addition, the exosomes may be comprised at a concentration of 1 to 50 μL/ml.

The “stem cells” in the present invention refer to cells of various origins as pluripotent cells that can differentiate into various tissue cells under appropriate conditions in an undifferentiated state.

The stem cells may be bone marrow-derived stem cells, cord blood-derived stem cells or adipose-derived stem cells. In an exemplary embodiment of the present invention, the stem cells may be adipose-derived stem cells. In addition, the bone marrow-derived stem cells, cord blood-derived stem cells, or adipose-derived stem cells may be human- or animal-derived stem cells.

The “adipose-derived stem cells” in the present invention are stem cells derived from adipose tissue and refer to cells having multipotency and self-renewal ability, and the adipose-derived stem cells of the present invention may be obtained through liposuction and various surgical procedures, but are not limited thereto.

In an exemplary embodiment of the present invention, the exosomes means being able to be transformed to express ligands that bind to receptors of colorectal cancer cell in a gene expressed in exosomes of host cells such as stem cells to act as a target for colorectal cancer cells.

Furthermore, in an exemplary embodiment of the present invention, the exosomes may be transfected with a nucleic acid capable of suppressing the expression of a gene inducing colorectal cancer.

The “transfection” in the present invention refers to a method for mutating the genetic traits of cells by directly introducing DNA or RNA into animal cells, and as the transfection method, a method known in the art, for example, calcium phosphate transfection, lipofection, electroporation, microinjection, and microprojectile bombardment may be used, but is not limited thereto. In the case of eukaryotes, it is possible to use methods of treating cells by mixing DNA with a commercial reagent, such as DNA calcium phosphate precipitation, or lipofection and PEI.

The “nucleic acid” in the present invention means being inserted into a gene that is expressed in the extracellular vesicles of stem cells to comprise a gene capable of encoding a peptide that binds to receptors of colorectal cancer cells when expressed in extracellular vesicles.

The “nucleic acid” in the present invention is a nucleic acid capable of suppressing the expression of a gene inducing colorectal cancer, and the expression of a gene inducing colorectal cancer may be suppressed by a method of binding to a nucleic acid that express oncogenes or directly binding to oncogenes to suppress expression.

The “nucleic acid” in the present invention may be one or more nucleic acids selected from the group consisting of miRNA, siRNA, shRNA, antisense RNA and ribozymes, and in an exemplary embodiment of the present invention, the “nucleic acid” comprises one or more nucleic acids selected from the group consisting of miRNA and siRNA.

Further, still another aspect of the present invention provides a method for preparing a pharmaceutical composition for preventing or treating colorectal cancer, the method comprising: (a) introducing a gene expressing a ligand that binds to receptors of colorectal cancer cells into a gene that is expressed in extracellular vesicles of adipose-derived stem cells: (b) obtaining extracellular vesicles by culturing the gene-introduced cells in a medium; and (c) transfecting the resulting extracellular vesicles with a nucleic acid capable of suppressing the expression of a gene inducing colorectal cancer.

The “extracellular vesicles” in the present invention may be exosomes, ectosomes or microvesicles, but in an exemplary embodiment of the present invention, the “extracellular vesicles” may be exosomes.

The “medium” in the present invention may be an adipose-derived stem cell culture medium. In an exemplary embodiment of the present invention, the adipose-derived stem cell culture medium may be a DMEM high low glucose medium or an FBS-free DMEM low glucose medium in which inactivated fetal bovine serum (FBS) and penicillin-streptomycin (P/S) are added for adipose-derived stem cells.

The “prevention” in the present invention means all actions that suppress colorectal cancer or delay the onset of colorectal cancer by administering the pharmaceutical composition according to the present invention.

The “treatment” in the present invention means all actions that ameliorate or beneficially change symptoms caused by colorectal cancer by administering the pharmaceutical composition according to the present invention.

The “colorectal cancer” in the present invention means malignant tumors composed of cancer cells generated in the large intestine. Pathologically, colorectal cancer is mostly adenocarcinoma that develops in the form of a polyp, and cancers may occur due to a carcinoid, lymphoma, and the like in rare cases. Colorectal cancer is divided into colon cancer and rectal cancer.

### Advantageous Effects

A pharmaceutical composition comprising extracellular vesicles transformed with a recombinant expression vector in which a gene expressing a ligand that binds to receptors of colorectal cancer cells is inserted into a gene that is expressed in extracellular vesicles of the stem cells of the present invention can be utilized as a drug delivery material and specifically act on colorectal cancer by selectively binding to colorectal cancer cells which are target cells. Therefore, the target exosomes can be applied in the field of colorectal cancer treatment, particularly as a clinical application technique.

### DESCRIPTION OF DRAWINGS

FIG. 1 is a schematic view illustrating how exosomes bind to cancer cells, which are target cells, by expressing a targeting ligand in exosomes through an expression vector. Upon treatment with exosomes targeting colorectal cancer, RNAs that suppress oncogenes can be inserted into the exosomes to be efficiently delivered to colorectal cancer cells rather than normal cells, thereby suppressing colorectal cancer cells.

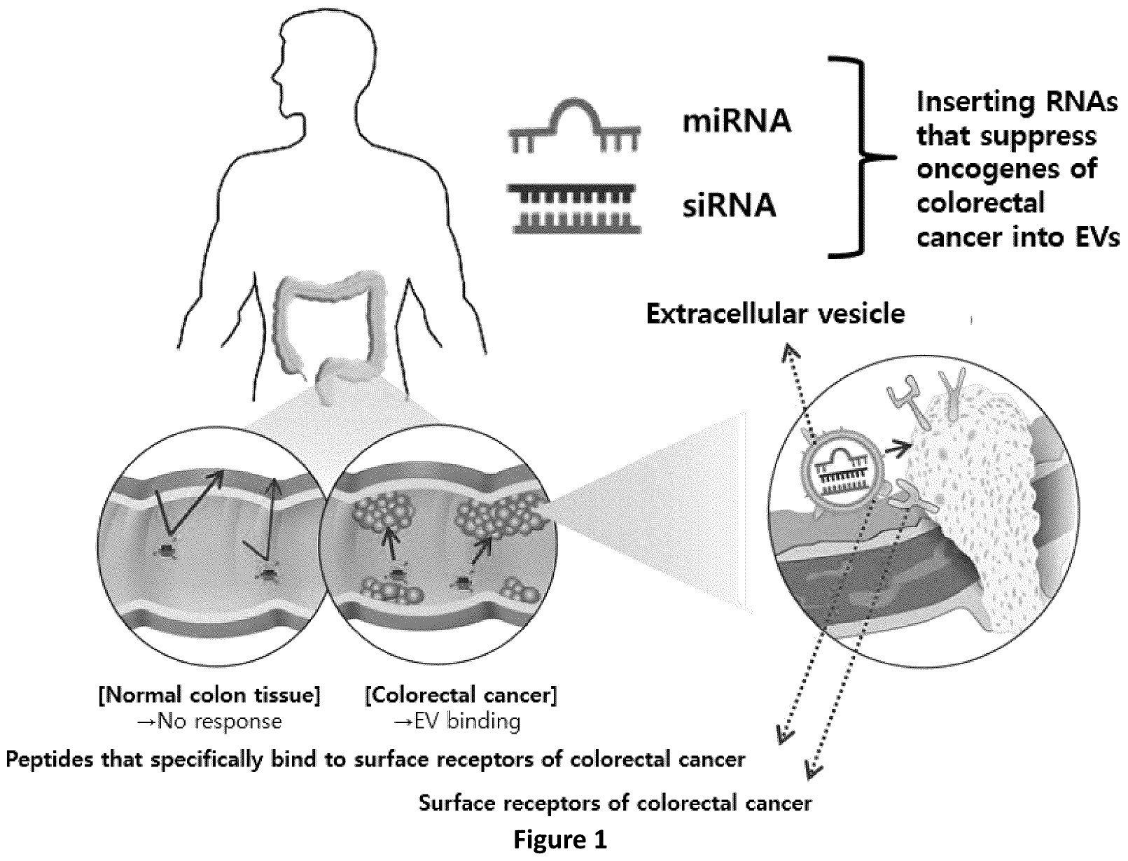

FIG. 2A illustrates the vector configuration in pDisplay for the expression of the recombinant protein of the present invention, and FIG. 2B illustrates the pattern according to the expression of the vector.

FIG. 3 illustrates the results of analysis using transmission electron microscopy (TEM) and Western blot for the structural analysis of exosomes.

FIG. 4 illustrates the results of real-time PCR analysis of HCT29, which is a colorectal cancer cell line. The vertical axis of the graph means relative density.

FIG. 5 illustrates the results showing cell migration according to the wound healing assay of exosomes.

FIG. 6 illustrates the expression levels of E-cad, snail, and PIN1, which are factors involved in the epithelial to mesenchymal transition (EMT) mechanism, in HCT116, which is a colorectal cancer cell line.

FIG. 7 illustrates the expression levels of E-cad, N-cad, vimentin, snail, and PIN1, which are factors involved in the epithelial to mesenchymal transition (EMT) mechanism, in a colorectal cancer animal model in which HCT116, a colorectal cancer cell line, is transplanted into a BALB/C nude mouse.

FIG. 8 illustrates the appearance observation results of a colorectal cancer animal model constructed by transplanting HCT116, a colorectal cancer cell line, into a BALB/C nude mouse.

FIG. 9 illustrates the tumor volume and body weight in a colorectal cancer animal model constructed by transplanting the colorectal cancer cell line HCT116 into a BALB/C nude mouse.

### MODES OF THE INVENTION

Hereinafter, the present invention will be described in more detail through examples. These examples are only for exemplifying the present invention, and it will be apparent to those of ordinary skill in the art that the scope of the present invention is not construed as being limited by these examples.

### Example 1. Production and Confirmation of Target Exosomes Derived from Adipose-Derived Stem Cells

Exosomes were extracted using adipose-derived stem cells to produce target exosomes that specifically act on colorectal cancer according to the present invention. For reference, in the present invention, adipose-derived stem cells were used as donor cells for producing target exosomes, but are not limited thereto because immune cells such as NK cells can also be used.

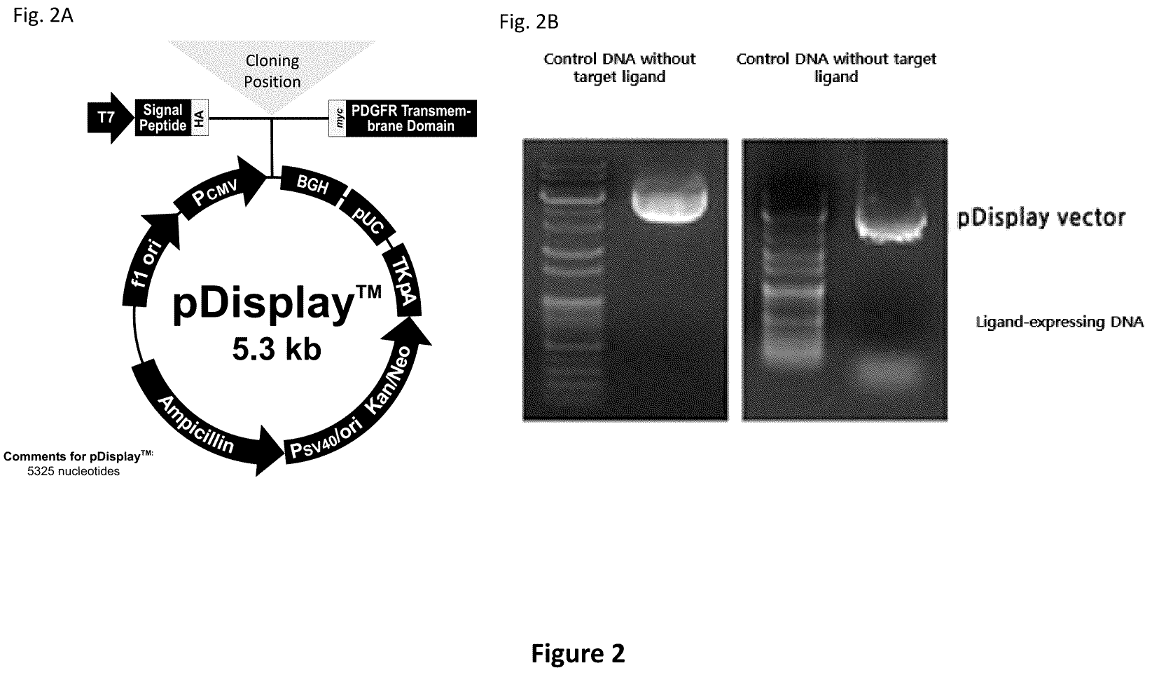

Adipose-derived stem cells were transduced with a base sequence encoding a colorectal cancer cell targeting ligand (ACG TGG TAT AAA ATC GCG TTT CAG CGC AAC CGA AAA) in a pDisplay vector (Invitrogen) using a transfection reagent (Lipofectamine, Invitrogen) according to the manufacturer's instructions. After cloning the vector, a cloned result of the corresponding vector was confirmed (FIG. 2), and target exosomes were produced by inserting the vector into adipose-derived stem cells.

To extract the produced target exosomes, adipose-derived stem cells were cultured in a DMEM low glucose medium supplemented with 10% inactivated fetal bovine serum (FBS) and 1% penicillin-streptomycin (P/S) under conditions of 37° ° C. and 5% CO₂.

After 24 hours, the medium was exchanged with an FBS-free DMEM low glucose medium, and after 24 hours after the medium exchange, the adipose-derived stem cell culture medium was harvested and centrifuged under 1000 rpm conditions by differential centrifugation to remove the cells.

Colorectal cancer target exosomes were obtained from the culture medium from which the cells were removed using a thermo exosome extraction kit (Thermo Fisher Scientific) according to the manufacturer's instructions.

Here, the target exosomes obtained through the above process were transfected with PIN-siRNA using an Exo-Fect kit (SBI System Biosciences), and through this, exosomes that can target colorectal cancer receptors were additionally produced.

### Example 2: Characterization of Target Exosomes Derived from Adipose-Derived Stem Cells

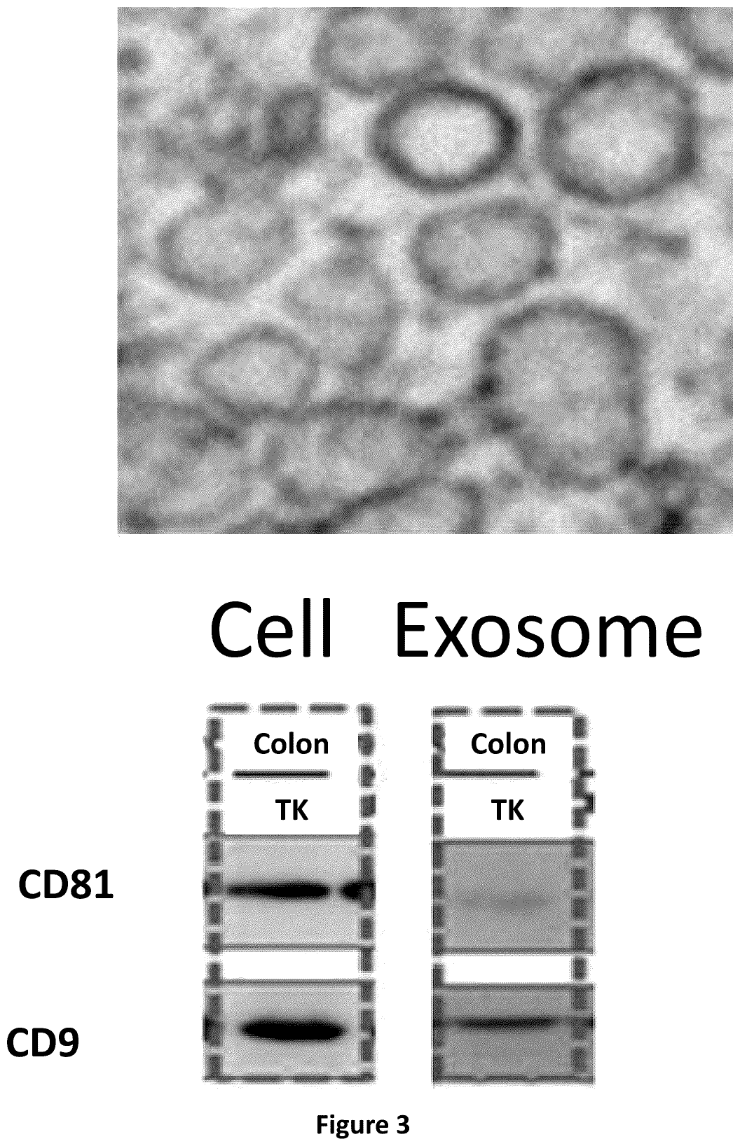

Transmission electron microscopy (TEM) and Western blot were used to analyze the structure of the target exosomes that specifically act on colorectal cancer according to the present invention. The TEM analysis and Western blot results for exosomes are illustrated in FIG. 3.

It could be confirmed that the exosomes isolated from the adipose-derived stem cell culture medium had a nanometer-sized spherical structure. In addition, as the expression of exosome surface markers CD81 and CD9 was confirmed using Western blot, it can be seen that the isolated exosomes have typical exosome characteristics (FIG. 3).

### Example 3. Real-Time PCR Analysis of Target Exosomes Derived from Adipose-Derived Stem Cells

In order to confirm the difference in expression by colorectal cancer cell line, genes to be inserted into colorectal cancer cell lines and colorectal cancer target exosomes were selected. HCT116 and HCT29 were selected as colorectal cancer cell lines, and PIN1, which is known to promote EMT in colorectal cancer, was selected as a gene to be inserted into target exosomes. Meanwhile, cancer cells are known to undergo migration and infiltration by transforming epithelial cells characteristics into those of mesenchymal cells through the process of the epithelial to mesenchymal transition (EMT).

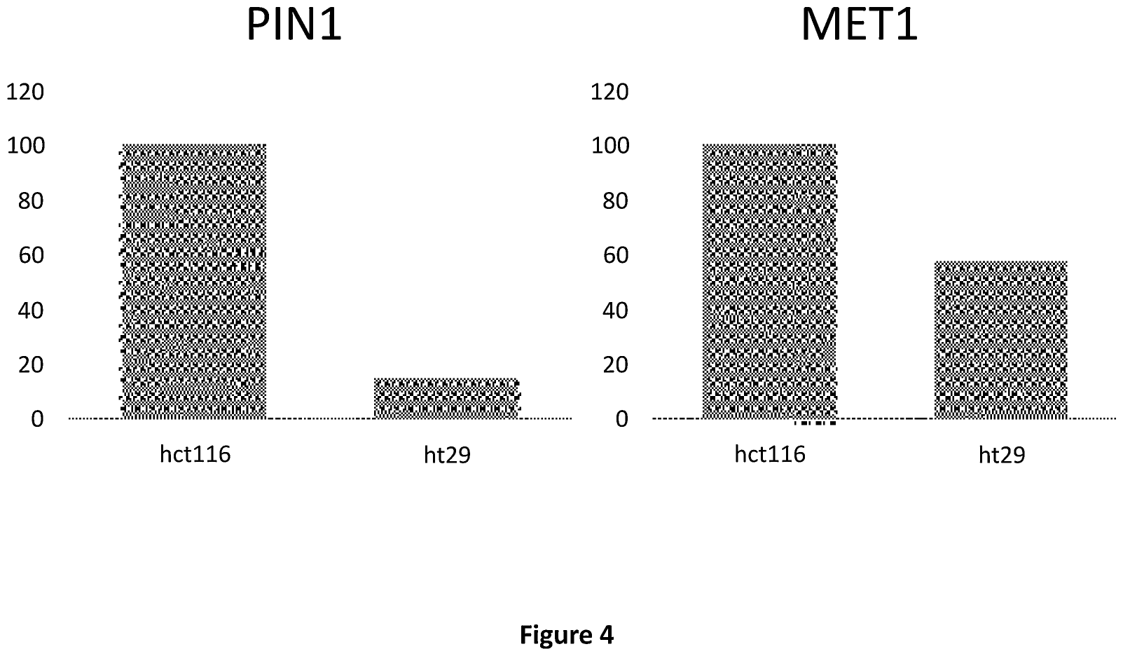

For real-time PCR analysis, 10 ng of RNA was converted into a first cDNA strand using specific primer using a cDNA kit (TOYOBO), and then using SYBR green (TOYOBO), real-time PCR was performed using a thermo PCR machine under the conditions of: 95ºC for 20 sec, 95ºC for 10 min (1cycle), 95° C. for 10 sec, 60° ° C. for 1 min (40 cycles), 95° C. for 15 sec, 60ºC for 1 min, 95° C. for 15 sec (1cycle), and then the results were observed (FIG. 4).

As a result, as illustrated in FIG. 4, it could be confirmed that the relative density values of EMT cells (MET1) and PIN1 tended to significantly decrease in HCT29 as a colorectal cancer cell line.

### Example 4. Evaluation of Tumorigenesis-Suppressing Ability of Target Exosomes Derived from Adipose-Derived Stem Cells

In order to observe the metastatic potential of the cells, a HCT116 colorectal cancer cell line was treated to perform wound healing assay. First, exosomes (TK-CT) as a control, target exosomes (TK), PIN-siRNA-loaded (siPIN) exosomes (siPIN TK-CT) and PIN-siRNA-loaded (siPIN) target exosomes (siPIN TK) were prepared.

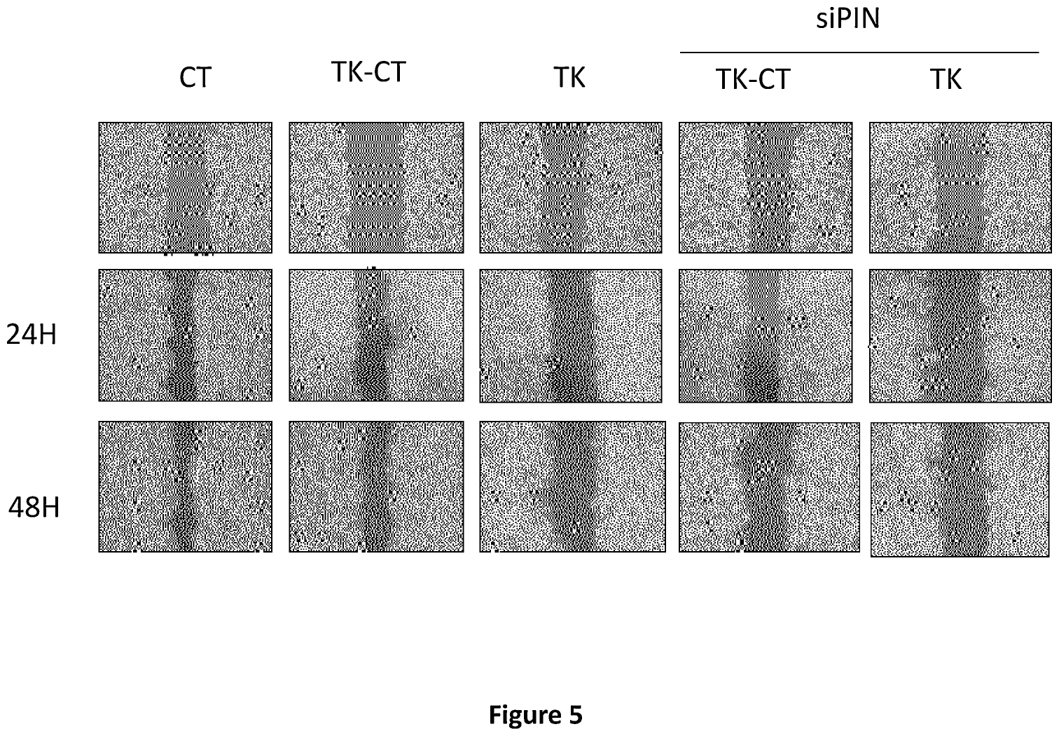

As a result of the experiment, it could be confirmed that when wound healing assay (24H and 48H) was performed, cell migration was suppressed in the PIN-siRNA-loaded (siPIN) exosomes (siPIN TK-CT) and the PIN-siRNA-loaded (siPIN) target exosomes (siPIN TK) (FIG. 5).

### Example 5. Efficacy Evaluation of Adipose-Derived Stem Cell-Derived Target Exosomes in Colorectal Cancer Cell Line HCT116

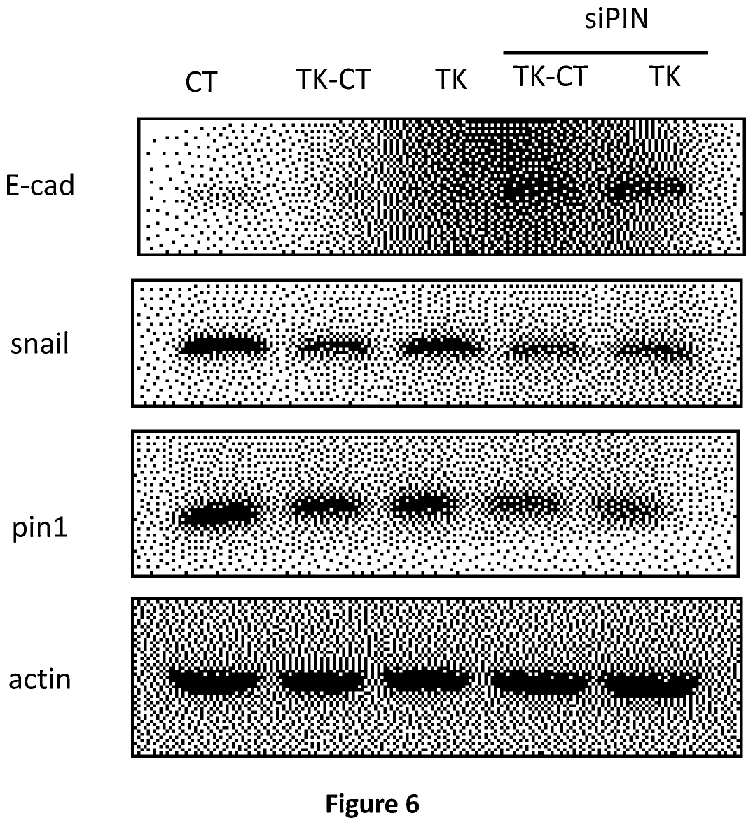

In order to evaluate the anti-cancer efficacy of colorectal cancer-targeting exosomes derived from adipose-derived stem cells cloned with the recombinant vector according to the present invention compared to exosomes derived from adipose-derived stem cells, HCT116, which is a colorectal cancer cell line, was treated with exosomes (TK-CT) as a control, target exosomes (TK), PIN-siRNA-loaded (si-pin) exosomes (si-pin TK-CT) and PIN-siRNA-loaded (si-pin) target exosomes (si-pin TK). Changes were observed by comparing the expression levels of E-cad, snail and PIN1, which are factors involved in the epithelial to mesenchymal transition (EMT) mechanism (FIG. 6).

As a result, as illustrated in FIG. 6, it could be confirmed that in PIN-siRNA-loaded (si-pin) exosomes (si-pin TK-CT) and PIN-siRNA-loaded (si-pin) target exosomes (si-pin TK), the expression levels of E-cad was increased and the expression levels of Snail and PIN1 were decreased to suppress EMT.

### Example 6. Efficacy Evaluation of Target Exosomes Derived from Adipose-Derived Stem Cells Using Colorectal Cancer Animal Model Following Transplantation of Colorectal Cancer Cell Line HCT116

After colorectal cancer was induced by transplanting HCT116, a colorectal cancer cell line, into a BALB/C nude mouse (in vivo) at 5×10⁵, the present inventors attempted to confirm the efficacy of treating colorectal cancer using exosomes derived from adipose-derived stem cells.

After each of exosomes (tk-ct) as a control, target exosomes (tk), PIN-siRNA-loaded (si-PIN1) exosomes (si-PIN1tk-ct) and PIN-siRNA-loaded (si-PIN1) target exosomes (si-PIN1tk) was injected into the tumor model constructed above by a tail vein injection method for 2 weeks, mice were sacrificed to confirm the effect of the exosomes on the anti-tumor effect for colorectal cancer.

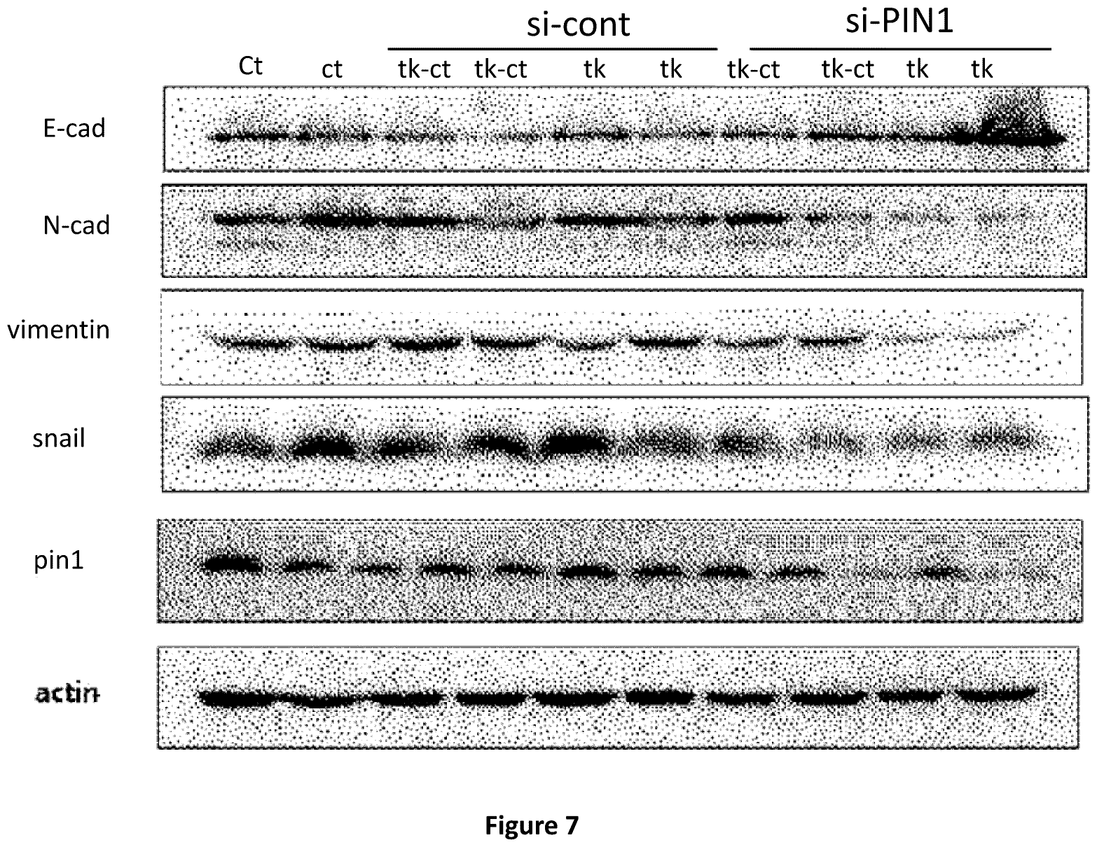

Changes were observed by comparing the expression levels of E-cad, N-cad, vimentin, snail and PIN1, which are factors involved in the epithelial to mesenchymal transition (EMT) mechanism (FIG. 7). As a result, as illustrated in FIG. 7, it could be confirmed that in PIN-siRNA-loaded (si-PIN1) exosomes (si-PIN1 tk-ct) and PIN-siRNA-loaded (si-PIN1) target exosomes (si-PIN1 tk), the expression levels of E-cad was increased and the expression levels of N-CAD, vimentin, snail and PIN1 were decreased to suppress EMT.

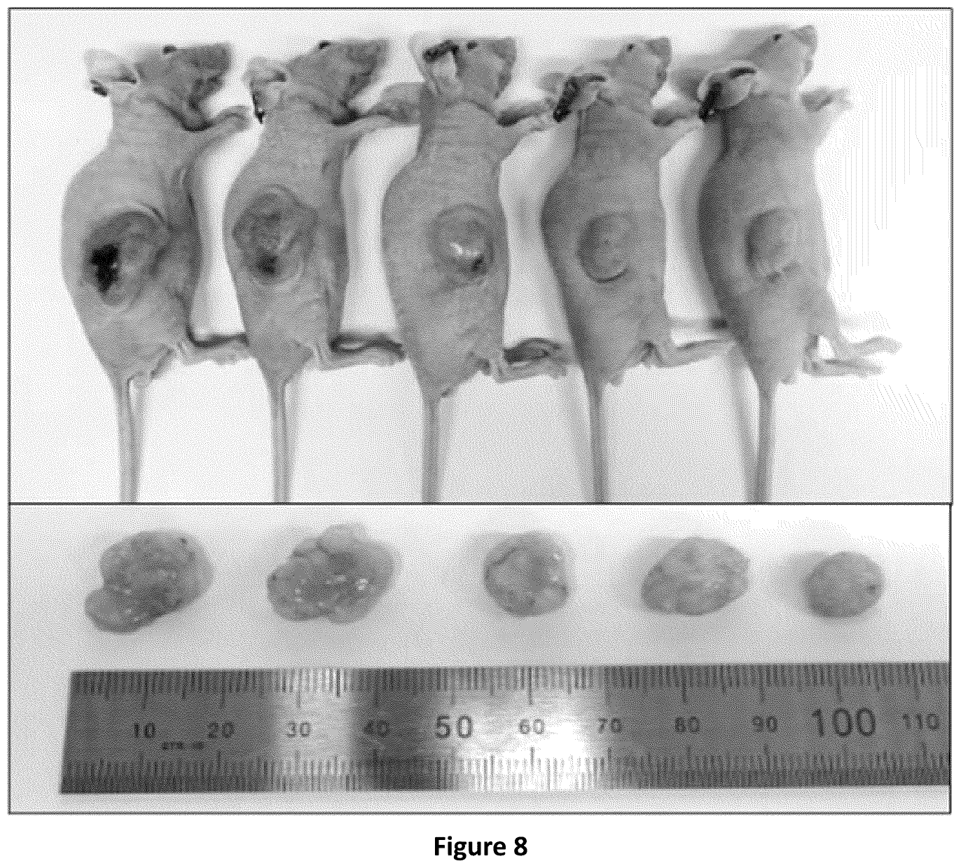

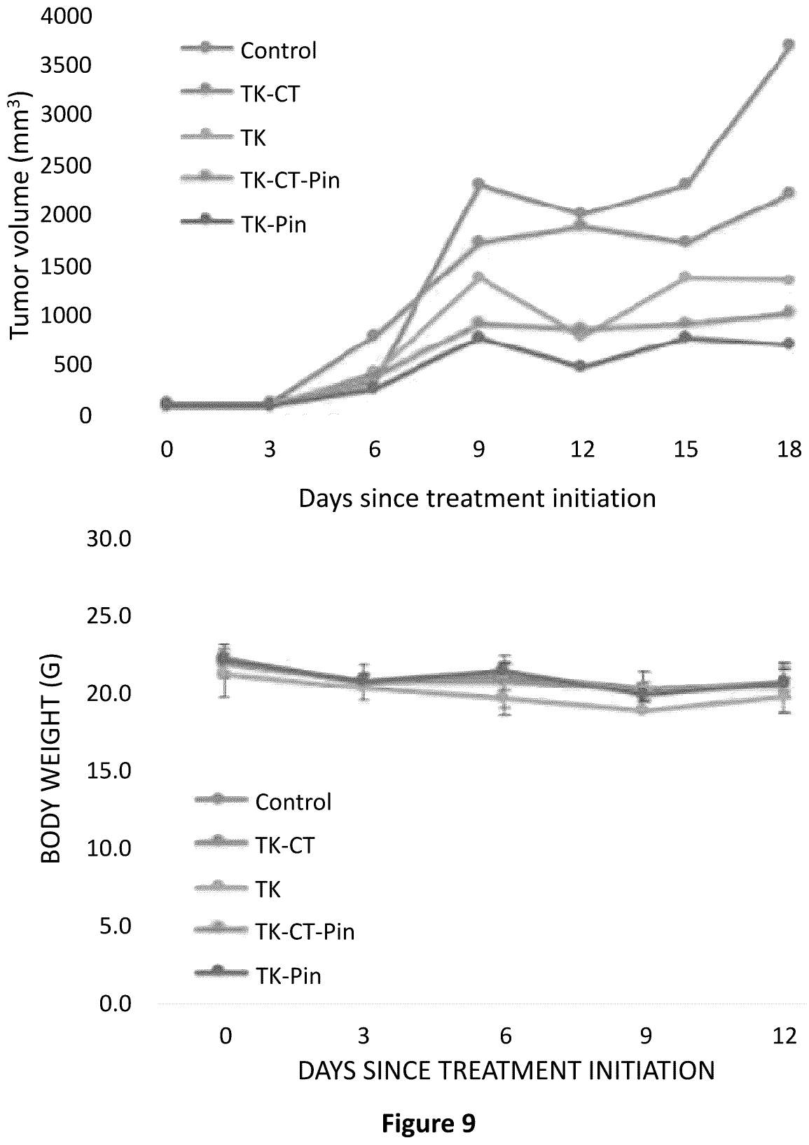

Furthermore, even by the appearance observation results in the colorectal cancer animal model constructed by transplanting the colorectal cancer cell line HCT116 into the BALB/C nude mouse (in vivo) at 5×10⁵, as illustrated in FIGS. 8 and 9, it was confirmed that the tumor size tended to be suppressed in the group treated with PIN-siRNA-loaded (siPIN) exosomes (TK-CT-Pin) and PIN-siRNA-loaded (siPIN) target exosomes (TK-Pin) (FIGS. 8 and 9).

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

1. A method for treating colorectal cancer, comprising a step of administering a composition comprising extracellular vesicles transformed with a recombinant expression vector in which a gene expressing a ligand that binds to receptors of colorectal cancer cells is inserted into a gene that is expressed in extracellular vesicles of stem cells.

2. The method of claim 1, wherein the ligand that binds to receptors of colorectal cells is represented by the nucleic acid sequence of SEQ ID NO:1.

3. The method of claim 1, wherein the extracellular vesicles are exosomes.

4. The method of claim 3, wherein the exosomes are transfected with a nucleic acid capable of suppressing the expression of a gene inducing colorectal cancer.

5. The method of claim 4, wherein the nucleic acid is one or more nucleic acids selected from the group consisting of miRNA, siRNA, antisense RNA and ribozymes.

6. The method of claim 3, wherein the exosomes are included at a concentration of 1 to 50 μL/ml

7. The method of claim 1, wherein the stem cells are bone marrow-derived stem cells, cord blood-derived stem cells or adipose-derived stem cells.

8. The method of claim 7, wherein the bone-marrow derived stem cells, cord blood-derived stem cells or adipose-derived stem cells are human- or animal-derived stem cells.

9. A method for preparing a pharmaceutical composition for treating colorectal cancer, the method comprising:
(a) introducing a gene expressing a ligand that binds to receptors of colorectal cancer cells into a gene that is expressed in extracellular vesicles of adipose-derived stem cells;
(b) obtaining extracellular vesicles by culturing the gene-introduced cells in a medium; and
(c) transfecting the resulting extracellular vesicles with a nucleic acid capable of suppressing the expression of a gene inducing colorectal cancer.

10. The method of claim 9, wherein the extracellular vesicles are exosomes.

11. A pharmaceutical composition for treating colorectal cancer, comprising extracellular vesicles transformed with a recombinant expression vector in which a gene expressing a ligand that binds to receptors of colorectal cancer cells is inserted into a gene that is expressed in extracellular vesicles of stem cells.
